(19) United States
(12) Patent Application Publication
(10) Pub. No.: US 2015/0139987 A1
(43) Pub. Date: May 21, 2015

(54) TREATMENT OF HOMOZYGOUS FAMILIAL HYPERCHOLESTEROLEMIA

(71) Applicant: CymaBay Therapeutics, Inc., Newark, CA (US)

(72) Inventors: Robert L. Martin, San Ramon, CA (US); Charles A. McWherter, Oakland, CA (US); Patrick J. O'Mara, San Ramon, CA (US)

(73) Assignee: CymaBay Therapeutics, Inc., Newark, CA (US)

(21) Appl. No.: 14/541,835

(22) Filed: Nov. 14, 2014

Related U.S. Application Data
(60) Provisional application No. 61/906,837, filed on Nov. 20, 2013.
(60) Provisional application No. 61/942,438, filed on Feb. 20, 2014.
(60) Provisional application No. 61/974,816, filed on Apr. 3, 2014.
(60) Provisional application No. 61/974,725, filed on Apr. 3, 2014.
(60) Provisional application No. 61/942,941, filed on Feb. 21, 2014.
(60) Provisional application No. 61/974,785, filed on Apr. 3, 2014.

(51) Int. Cl. A61K 31/192; C12N 15/113; A61K 39/395; A61K 31/4468

(52) U.S. Cl. CPC A61K 31/192; A61K 31/4468; C12N 15/113; A61K 39/3955; C12N 2310/11; C12N 2320/31
USPC 424/133.1; 514/571; 514/325; 514/44 A; 424/142.1

(57) ABSTRACT

Treatment of homozygous familial hypercholesterolemia by administration of (R)-2-(4-((2-ethoxy-3-(4-(trifluoromethyl)phenoxy)propyl)thio)-2-methylphenoxy)acetic acid or a salt thereof, optionally in combination with an MTP inhibitor, an apoB-100 synthesis inhibitor, or a PCSK9 inhibitor.

### CROSS-REFERENCE TO RELATED APPLICATIONS

This application claims the priority under 35 USC 119(e) of the following six provisional applications: App. No. 61/906,837, filed Nov. 20, 2013; App. No. 61/942,438, filed Feb. 20, 2014; App. No. 61/974,816, filed Apr. 3, 2014; and App. No. 61/974,725, filed Apr. 3, 2014; all entitled “Treatment of dyslipidemias and related conditions”; and App. No. 61/942,941, filed Feb. 21, 2014; and App. No. 61/974,785, filed Apr. 3, 2014; both entitled “Treatment of homozygous familial hypercholesterolemia”. The disclosures of each of these six applications are incorporated into this application by reference.

### BACKGROUND OF THE INVENTION

1. Field of the Invention

This invention relates to the treatment of homozygous familial hypercholesterolemia.

2. Description of the Related Art

Homozygous Familial Hypercholesterolemia

Dyslipidemia is the presence of an abnormal amount of lipids (e.g. cholesterol and/or fat) in the blood. In developed countries, most dyslipidemias are hyperlipidemias; that is, an elevation of lipids/lipoproteins in the blood—the term hyperlipidemia is often used to include hyperlipoproteinemia. Hyperlipidemias include hypercholesterolemia (elevated cholesterol) and hyperglyceridemia (elevated glycerides), with hypertriglyceridemia (elevated triglycerides (TGs)) as a subset of hyperglyceridemia: combined hyperlipidemia refers to an elevation of both cholesterol and triglycerides. Hyperlipoproteinemia refers to the presence of elevated lipoproteins (usually low-density lipoproteins (LDL) unless otherwise specified), with hyperchylomicronemia (elevated chylomicrons) as a subset of hyperlipoproteinemia. Mixed hyperlipidemia (combined hyperlipidemia) refers to elevated TGs and LDL. Familial (i.e., genetically-caused) hyperlipidemias are classified according to the Fredrickson classification, which is based on the pattern of lipoproteins on electrophoresis or ultracentrifugation: Type II includes familial hypercholesterolemia (FH, Type IIa) and familial combined hyperlipidemia (Type IIb). Hyperlipidemias such as hypercholesterolemia, mixed hyperlipidemia, and hyperlipoproteinemia generally involve elevated LDL and low-density lipoprotein cholesterol (LDL-C, “bad cholesterol”), and are frequently accompanied by decreased high density lipoproteins (HDL) and high-density lipoprotein cholesterol (HDL-C, “good cholesterol”).

FH is a genetic disorder characterized by high cholesterol levels, specifically very high levels of LDL-C, in the blood, and early cardiovascular disease (CVD). The high cholesterol levels in FH are less responsive to the kinds of cholesterol control methods that are usually more effective in people without FH (such as dietary modification and statins), because the body's underlying biochemistry is slightly different in these genetically-linked conditions and the body is often overwhelmed by the magnitude of the abnormal levels of lipids. However, treatment (including higher statin doses) can often provide benefit. Many patients with FH have mutations in the LDLR gene that encodes the LDL receptor protein, which normally removes LDL from the circulation, or apolipoprotein B (apoB), which is the part of LDL that binds with the receptor, both types of mutations leading to elevated LDL-C; mutations in other genes that affect LDL receptor function do occur, but are less frequent. Patients who have one abnormal copy (heterozygous) of the LDLR gene may have premature CVD at the age of 30 to 40. Patients who have two abnormal copies (homozygous) may experience severe CVD in childhood, and without treatment may experience myocardial infarction, ischemic stroke, and death by around the age of 30. Heterozygous FH (HeFH) is a common genetic disorder, inherited in an autosomal dominant pattern, occurring in 1 in 500 people in most countries; homozygous FH (HoFH) is much rarer, occurring in 1 in 1,000,000 people. HeFH is normally treated with statins, bile acid sequestrants or other hypolipidemic agents that lower cholesterol levels. New cases are generally offered genetic counselling. HoFH often does not respond to medical therapy and may require other treatments, including LDL apheresis (removal of LDL in a method similar to dialysis) and occasionally liver transplantation. Therapies such as statins work primarily by up-regulating liver LDL receptor expression, thereby increasing LDL receptor-mediated clearance of lipids. Thus patients with HoFH (and severe HeFH), who lack functional LDL receptor activity, will generally respond poorly to such therapies. Subjects with receptor-defective HoFH have some residual LDL receptor activity and may see modest reductions in LDL-C with maximal conventional therapy; while subjects with receptor-negative HoFH will generally not benefit significantly. According to Moorjani et al., “Mutations of low-density-lipoprotein-receptor gene, variation in plasma cholesterol, and expression of coronary heart disease in homozygous familial hypercholesterolemia”, Lancet, 341(8856), 1303-1306 (1993), and Goldstein et al, “The LDL Receptor”, Arterioscler. Thromb. Vasc. Biol., 29, 431-438 (2009), patients with receptor-negative HoFH have higher levels of LDL-C (often >750 mg/dL) and develop severe CVD at an earlier age than patients with receptor-defective HoFH (LDL-C levels 400-600 mg/dL). According to Winters, “Low-density lipoprotein apheresis: principles and indications”, Sem. Dialysis, 25(2), 145-151 (2012), apheresis reduces CVD events in patients with HoFH. Considerable evidence in other hypercholesterolemic conditions supports the causality of elevated LDL-C in atherosclerotic CVD and the link between lowering LDL-C and reduction in CVD events; so that reductions in LDL-C can be expected to reduce the risk of CVD in HoFH patients.

Recent Developments in Treatments for Homozygous Familial Hypercholesterolemia

Treatments recently approved for HoFH in the US fall into two classes: microsomal triglyceride transfer protein (MTP) inhibitors and apolipoprotein B-100 (apoB-100) synthesis inhibitors. A third class, proprotein convertase subtilisin-like kexin type 9 (PCSK9) inhibitors, is under development for hypercholesterolemia and is considered to have potential efficacy in HoFH.

MTP Inhibitors

Lomitapide (INN, USAN) is the compound of the formula

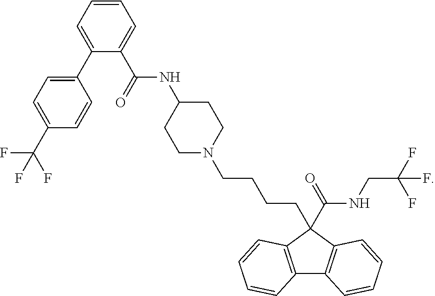 [SMILES: O=C(NC1CCN(CCCCC2(C(=O)NCC(F)(F)F)c3ccccc3-c3ccccc32)CC1)c1ccccc1-c1ccc(C(F)(F)F)cc1]

Lomitapide has the chemical name N-(2,2,2-trifluoroethyl)-9-(4-(4-(4′-(trifluoromethyl)-[1,1′-biphenyl]-2-ylcarboxamido)piperidin-1-yl)butyl)-9H-fluorene-9-carboxamide [IUPAC name as generated by CHEMDRAW ULTRA 12.0]. Lomitapide and its synthesis, formulation, and use is disclosed in, for example, U.S. Pat. No. 5,712,279 (the compound of Example 73 and claim 13) and U.S. Pat. No. 5,739,135 (the compound of claim 23, generically).

Lomitapide is an orally active potent inhibitor of microsomal triglyceride transfer protein (MTP), which is necessary for very low-density lipoprotein (VLDL) assembly and secretion from the liver; and is also a selective inhibitor of the secretion of apoB-containing lipoproteins. MTP is also expressed in intestinal enterocytes where it mediates both triglyceride absorption and chlymicron secretion into the blood. According to the patents mentioned above, lomitapide is suggested as being useful “for preventing, inhibiting or treating atherosclerosis, pancreatitis or obesity” and “for lowering serum lipid levels, cholesterol and/or triglycerides, or inhibiting and/or treating hyperlipemia, hyperlipidemia, hyperlipoproteinemia, hypercholesterolemia, and/or hypertriglyceridemia”. A six-patient dose-escalation study in patients with HoFH is described in Cuchel et al., “Inhibition of Microsomal Triglyceride Transfer Protein in Familial Hypercholesterolemia”, N. Engl. J. Med., 356(2), 148-156 (2007). Patients in that study were treated with doses of 0.03, 0.1, 0.3, and 1.0 mg/kg/day lomitapide mesylate (i.e. about 2, 7, 20, and 70 mg/day for a 70 kg person), each for 4 weeks. U.S. Pat. No. 7,932,268 discloses and claims treatment of hyperlipidemia and hypercholesterolemia by stepwise dose escalation of an MTP inhibitor, including lomitapide: the dose escalation is said to minimize the adverse reactions to the MTP inhibitor administration. In the Phase III clinical trial of lomitapide, 29 subjects with HoFH were treated with lomitapide mesylate in doses of 5 mg/day for 2 weeks; 10, 20 and 40 mg/day each for 4 weeks; and 60 mg/day for 12 weeks; then continued on their maximum tolerated dose not exceeding 60 mg/day for 52 weeks. Subjects were instructed to maintain a low-fat diet (<20% energy from fat) and to take dietary supplements that provided approximately 400 IU vitamin E, 210 mg α-linolenic acid, 200 mg linoleic acid, 110 mg eicosapentaenoic acid, and 80 mg docosahexaenoic acid per day.

Combination therapy with MTP inhibitors such as lomitapide “for lowering serum lipids, cholesterol and/or triglycerides and thereby inhibiting atherosclerosis” is disclosed in U.S. Pat. No. 5,883,109 (“Cholesterol lowering drugs or drugs which are inhibitors of cholesterol biosynthesis which may be used in the method of the invention in combination with the MTP inhibitor include HMG CoA reductase inhibitors, squalene synthetase inhibitors, fibric acid derivatives, bile acid sequestrants, probucol, niacin, niacin derivatives, neomycin, aspirin, and the like”). The use of MTP inhibitors such as lomitapide “for inhibiting or treating diseases associated with acid lipase deficiency” by administering an MTP inhibitor alone or in combination with another cholesterol lowering drug is disclosed in U.S. Pat. No. 6,066,653 (“The other cholesterol lowering drugs or delipidating drugs which may be used in the method of the invention include HMG CoA reductase inhibitors, squalene synthetase inhibitors, fibric acid derivatives, bile acid sequestrants, probucol, niacin, niacin derivatives and the like”). U.S. Pat. No. 7,932,268, mentioned previously, also discloses possible combination therapy with the MTP inhibitor and “other lipid modifying compounds”, including “HMG CoA reductase inhibitors, cholesterol absorption inhibitors, ezetimibe, squalene synthetase inhibitors, fibrates, bile acid sequestrants, statins, probucol and derivatives, niacin, niacin derivatives, PPAR alpha agonists, fibrates, PPAR gamma agonists, thiazolidinediones, and cholesterol ester transfer protein (CETP) inhibitors”.

Lomitapide mesylate is approved in the United States as the active compound in JUXTAPID, which is indicated as an adjunct to a low-fat diet and other lipid-lowering treatments, including LDL apheresis where available, to reduce LDL-C, total cholesterol (TC), apoB, and non-high-density lipoprotein cholesterol (non-HDL-C) in patients with HoFH. It is subject to a Risk Evaluation and Mitigation Strategy (REMS) because of the risk of hepatotoxicity. It is available in capsules containing 5, 10, and 20 mg lomitapide mesylate, and the maximum indicated daily dose is 60 mg, with reductions for certain concomitant medications or conditions. Because taking lomitapide with food may adversely affect gastrointestinal tolerability, JUXTAPID is labeled to be taken with water once/day at least 2 hours after the evening meal. Lomitapide mesylate is also approved in the European Union (under “exceptional circumstances”) as the active compound in LOJUXTA, which is indicated as an adjunct to a low-fat diet and other lipid-lowering medicinal products with or without LDL apheresis in adult patients with HoFH. The approval conditions for LOJUXTA state that genetic confirmation of HoFH should be obtained whenever possible, and other forms of primary hyperlipoproteinemia and secondary causes of hypercholesterolemia must be excluded.

From the Phase 3 trial, the most common adverse reactions to lomitapide were gastrointestinal, reported by 27 (93%) of 29 patients. Adverse events (AEs) reported by 8 (28%) or more patients in the trial included diarrhea (79%), nausea (65%), vomiting, dyspepsia and abdominal pain. Other common AEs, reported by 5 to 7 (17-24%) patients, included weight loss, abdominal discomfort, abdominal distension, constipation, flatulence, increased alanine aminotransferase, chest pain, influenza, nasopharyngitis, and fatigue. Five of the 29 patients discontinued the trial for AEs.

The US prescribing information for JUXTAPID contains a “black-box” warning: “WARNING: RISK OF HEPATOTOXICITY. JUXTAPID can cause elevations in transaminases. In the JUXTAPID clinical trial, 10 (34%) of the 29 patients treated with JUXTAPID had at least one elevation in alanine aminotransferase (ALT) or aspartate aminotransferase (AST) ≧3× upper limit of normal (ULN). There were no concomitant clinically meaningful elevations of total bilirubin, international normalized ratio (INR), or alkaline phosphatase [see Warnings and Precautions (5.1)]. JUXTAPID also increases hepatic fat, with or without concomitant increases in transaminases. The median absolute increase in hepatic fat was 6% after both 26 and 78 weeks of treatment, from 1% at baseline, measured by magnetic resonance spectroscopy. Hepatic steatosis associated with JUXTAPID treatment may be a risk factor for progressive liver disease, including steatohepatitis and cirrhosis [see Warnings and Precautions (5.1)]. Measure ALT, AST, alkaline phosphatase, and total bilirubin before initiating treatment and then ALT and AST regularly as recommended. During treatment, adjust the dose of JUXTAPID if the ALT or AST are ≧3× ULN. Discontinue JUXTAPID for clinically significant liver toxicity [see Dosage and Administration (2.4) and Warnings and Precautions (5.1)]. Because of the risk of hepatotoxicity, JUXTAPID is available only through a restricted program under a Risk Evaluation and Mitigation Strategy (REMS) called the JUXTAPID REMS PROGRAM [see Warnings and Precautions (5.2)].” JUXTAPID is contraindicated in pregnancy, concomitant administration of strong cytochrome P450 3A4 (CYP 3A4) inhibitors (weak CYP 3A4 inhibitors are permitted with a limitation of the lomitapide mesylate dose to 30 mg/day), and in patients with moderate or severe hepatic impairment (Child-Pugh category B or C) and patients with active liver disease including unexplained persistent elevations of serum transaminases.

The risk of hepatotoxicity is probably linked to the mechanism of action, in which inhibition of MTP in the liver leads to the accumulation of hepatic fat (see above). Because of the risk of hepatotoxicity and the adverse reactions observed, and because the clinical studies of lomitapide have been in HoFH, its approved use is significantly restricted. Nonetheless, lomitapide is a potent MTP inhibitor with significant lipid lowering effects (reduction of LDL-C by up to 65% in healthy volunteers with hypercholesterolemia). It would be desirable to reduce the adverse reactions in treatment with lomitapide, thereby improving its safety profile.

Other orally-active MTP inhibitors under development include the enterocytic MTP inhibitor SLx-4090, phenyl 6-(4′-trifluoromethyl-6-methoxybiphenyl-2-ylcarboxamido)-1,2,3,4-tetrahydroisoquinoline-2-carboxylate, (see Kim et al., “A Small-Molecule Inhibitor of Enterocytic Microsomal Triglyceride Transfer Protein, SLx-4090: Biochemical, Pharmacodynamic, Pharmacokinetic, and Safety Profile”, J. Pharmacol. Exp. Ther., 337, 775-785 (2011)), and JTT-130, diethyl 2-({3-dimethylcarbamoyl-4-[(4′-trifluoromethylbiphenyl-2-carbonyl)amino]phenyl}-acetyloxymethyl)-2-phenylmalonate (see Mera et al., “JTT-130, a Novel Intestine-Specific Inhibitor of Microsomal Triglyceride Transfer Protein, Reduces Food Preference for Fat”, J. Diabetes Res., Article 83752 (2014) and Hata et al., “JTT-130, a Novel Intestine-Specific Inhibitor of Microsomal Triglyceride Transfer Protein, Suppresses Food Intake and Gastric Emptying with the Elevation of Plasma Peptide YY and Glucagon-Like Peptide-1 in a Dietary Fat-Dependent Manner”, J. Pharmacol. Exp. Ther., 336, 850-856 (2011)).

ApoB-100 Synthesis Inhibitors

Mipomersen (INN) is a synthetic phosphorothioate oligonucleotide, 20 nucleotides in length, of the sequence

(3′→5′)(P-thio)
(G-C-C-U-C-dA-dG-dT-dC-dT-dG-dC-dT-dT-dC-G-C-A-C-C)

where the modified nucleosides are: A=2′-O-(2-methoxyethyl)adenosine, C=2′-O-(2-methoxyethyl)-5-methylcytidine, G=2′-O-(2-methoxyethyl)guanosine, U=2′-O-(2-methoxyethyl)-5-methyluridine, and dC=2′-deoxy-5-methylcytidine. Mipomersen has the chemical name 2′-O-(2-methoxyethyl)-P-thioguanylyl-(3′→5′)-2′-O-(2-methoxyethyl)-5-methyl-P-thiocytidylyl-(3′→5′)-2′-O-(2-methoxyethyl)-5-methyl-P-thiocytidylyl-(3′→5′)-2′-O-(2-methoxyethyl)-5-methyl-P-thiouridylyl-(3′→5′)-2′-O-(2-methoxyethyl)-5-methyl-P-thiocytidylyl-(3′→5′)-2′-deoxy-P-thioadenylyl-(3′→5′)-2′-deoxy-P-thioguanylyl-(3′→5′)-P-thiothymidylyl-(3′→5′)-2′-deoxy-5-methyl-P-thiocytidylyl-(3′→5′)-P-thiothymidylyl-(3′→5′)-2′-deoxy-P-thioguanylyl-(3′→5′)-2′-deoxy-5-methyl-P-thiocytidylyl-(3′→5′)-P-thiothymidylyl-(3′→5′)-P-thiothymidylyl-(3′→5′)-2′-deoxy-5-methyl-P-thiocytidylyl-(3′→5′)-2′-O-(2-methoxyethyl)-P-thioguanylyl-(3′→5′)-2′-O-(2-methoxyethyl)-5-methyl-P-thiocytidylyl-(3′→5′)-2′-O-(2-methoxyethyl)-P-thioadenylyl-(3′→5′)-2′-O-(2-methoxyethyl)-5-methyl-P-thiocytidylyl-(3′→5′)-2′-O-(2-methoxyethyl)-5-methylcytidine [IUPAC name taken from the INN listing]. Mipomersen sodium is the nonadecasodium salt of mipomersen.

Mipomersen is an antisense oligonucleotide targeted to human messenger ribonucleic acid (mRNA) for apoB-100, the form of apoB produced in the liver and the principal apolipoprotein of LDL and its metabolic precursor, very-low-density-lipoprotein (VLDL). Mipomersen is complementary to the coding region of the mRNA for apoB-100, and binds by Watson and Crick base pairing. The hybridization of mipomersen to the cognate mRNA results in RNase H-mediated degradation of the cognate mRNA thus inhibiting translation of the apoB-100 protein. The in vitro pharmacologic activity of mipomersen was characterized in human hepatoma cell lines (HepG2, Hep3B) and in human and cynomolgus monkey primary hepatocytes. In these experiments, mipomersen selectively reduced apoB mRNA, protein, and secreted protein in a concentration- and time-dependent manner The effects of mipomersen were shown to be highly sequence-specific. The binding site for mipomersen lies within the coding region of the apoB mRNA at the position 3249-3268 relative to the published sequence in GenBank accession number NM_{—}000384.1. Mipomersen has an absorption time to maximum concentration after subcutaneous injection of 3-4 hours, a distribution half-life of about 2-5 hours, and an elimination half-life of 1-2 months, giving a steady-state plasma trough typically within 6 months. In dose-ranging trials, mipomersen sodium was dosed at 100 and 200 mg once/2 weeks and at 100, 200, 300, and 400 mg once/week. According to mipomersen's sponsor, efficacy increased with dose; but a “challenging” incidence of side effects was seen at the 300 and 400 mg doses, and the incidence of side effects was similar for both the 100 and 200 mg once/week doses, leading to the choice of 200 mg once/week as the Phase 3 trial dose.

Mipomersen sodium is approved in the United States as the active compound in KYNAMRO, which is indicated as an adjunct to lipid-lowering medications and diet to reduce LDL-C, apoB, TC, and non-HDL-C in patients with HoFH. It is subject to a REMS because of the risk of hepatotoxicity. It is available in prefilled syringes and vials containing 200 mg mipomersen sodium in 1 mL sterile aqueous solution, and the indicated weekly dose is 200 mg. The US Food & Drug Administration's “Orange Book” lists the following patents for KYNAMRO: U.S. Pat. Nos. 6,166,197, 6,222,025, 6,451,991, 7,015,315, 7,101,993, 7,407,943, and 7,511,131. All but U.S. Pat. No. 7,407,943 are said to have “drug substance” claims, generally directed to nucleotides and oligonucleotides with modified sugar residues; while U.S. Pat. No. 7,407,943 claims methods of inhibiting the expression of apoB, or decreasing serum cholesterol, lipoproteins, or serum triglycerides by administration of certain antisense oligonucleotides.

Mipomersen has been refused approval in the European Union, with the European Medicines Agency's Committee for Medicinal Products for Human Use (CHMP) noting that, although KYNAMRO was effective in reducing cholesterol levels in patients with HoFH and severe HeFH, there was concern about KYNAMRO's safety; in particular that: (a) a high proportion of patients stopped taking the medicine within two years, even in the restricted group of patients with HoFH, mainly due to side effects—this was considered an important limitation because KYNAMRO is intended for long-term treatment; (b) they were concerned by the potential long-term consequences of liver test results showing a build-up of fat in the liver and increased enzyme levels, and were not convinced that the sponsor had proposed sufficient measures to prevent the risk of irreversible liver damage; and (c) they were concerned that more cardiovascular events (problems with the heart and blood vessels) were reported in patients taking KYNAMRO than in patients taking placebo; so that this prevented the CHMP from concluding that KYNAMRO's intended cardiovascular benefit, in terms of reducing cholesterol levels, outweighed its potential cardiovascular risk.

KYNAMRO has been tested in 4 Phase 3 clinical trials: a pivotal trial in HoFH with 51 patients and 3 supportive trials, a severe hyperlipidemia trial (primarily HeFH) with 58 patients, an HeFH with coronary artery disease trial with 124 patients, and a high-risk coronary heart disease trial with 158 patients, with a combined open-label extension. All were randomized (2:1 mipomersen: placebo), double-blind, trials evaluating subcutaneous 200 mg mipomersen sodium once/week added to maximally-tolerated lipid-lowering therapy. Of the 390 patients dosed in the 4 trials, 28% of the mipomersen patients discontinued their trial, 18% for an adverse event (AE) or serious adverse event (SAE), 6% for patient withdrawal, and 4% for other reasons; while 7% of the placebo patients discontinued their trial, 2% for an adverse event or severe adverse event, 4% for patient withdrawal, and 1% for other reasons; while in the open-label extension, 55% of all patients and 61% of HoFH patients discontinued treatment, of which the majority of discontinuations were due to an AE or SAE. From these Phase 3 trials, the most common adverse reactions to mipomersen were injection site reactions (84% for mipomersen vs. 33% for placebo), flu-like symptoms (such as fatigue, fever, and chills) (30% vs. 16%), elevated serum aminotransferases (aspartate aminotransferase ≧3× upper limit of normal: 16% vs. 1%; alanine aminotransferase ≧3× upper limit of normal: 10% vs. 1%), hepatic steatosis, and headache and dizziness.

The US prescribing information for KYNAMRO contains a “black-box” warning: “WARNING: RISK OF HEPATOTOXICITY. KYNAMRO can cause elevations in transaminases. In the KYNAMRO clinical trial in patients with HoFH, 4 (12%) of the 34 patients treated with KYNAMRO compared with 0% of the 17 patients treated with placebo had at least one elevation in alanine aminotransferase (ALT) ≧3× upper limit of normal (ULN). There were no concomitant clinically meaningful elevations of total bilirubin, international normalized ratio (INR) or partial thromboplastin time (PTT) [see Warnings and Precautions (5.1)]. KYNAMRO also increases hepatic fat, with or without concomitant increases in transaminases. In the trials in patients with heterozygous familial hypercholesterolemia (HeFH) and hyperlipidemia, the median absolute increase in hepatic fat was 10% after 26 weeks of treatment, from 0% at baseline, measured by magnetic resonance imaging (MRI). Hepatic steatosis is a risk factor for advanced liver disease; including steatohepatitis and cirrhosis [see Warnings and Precautions (5.1)]. Measure ALT, AST, alkaline phosphatase, and total bilirubin before initiating treatment and then ALT, AST regularly as recommended. During treatment, withhold the dose of KYNAMRO if the ALT or AST are ≧3× ULN. Discontinue KYNAMRO for clinically significant liver toxicity [see Dosage and Administration (2.3) and Warnings and Precautions (5.1)]. Because of the risk of hepatotoxicity, KYNAMRO is available only through a restricted program under a Risk Evaluation and Mitigation Strategy (REMS) called the KYNAMRO REMS [see Warnings and Precautions (5.2)]. The safety and effectiveness of KYNAMRO have not been established in patients with hypercholesterolemia who do not have HoFH. The effect of KYNAMRO on cardiovascular morbidity and mortality has not been determined The use of KYNAMRO as an adjunct to LDL apheresis is not recommended.”

The risk of hepatotoxicity is probably linked to the mechanism of action, in which inhibition of apoB synthesis in the liver leads to the accumulation of hepatic fat (see above). Because of the risk of hepatotoxicity and the adverse reactions observed, and because the clinical studies of mipomersen have been in severe heterozygous FH and HoFH, its approved use is significantly restricted. Nonetheless, mipomersen is a potent inhibitor of apoB synthesis with significant lipid lowering effects (reduction of LDL-C by 25% when added to maximally tolerated lipid lowering medications in HoFH patients). It would be desirable to reduce the adverse reactions in treatment with mipomersen, thereby improving its safety profile.

Because of the significant adverse effects associated with both lomitapide and mipomersen, it would be desirable to develop an alternative that is effective in the treatment of HoFH but lacks these adverse effects.

PCSK9 Inhibitors

According to Manolis et al., “Novel Hypolipidemic Agents: Focus on PCSK9 Inhibitors”, Hosp. Chron., 9(1), 3-10 (2014), proprotein convertase subtilisin kexin type 9 (PCSK9), is a protein (serine protease) synthesized and secreted mainly by the liver which binds to hepatic LDL receptors. It regulates plasma LDL-C levels by diverting cell surface LDL receptors to lysosomes for degradation. In so doing, PCSK9 prevents the normal recycling of LDL receptors back to the cell surface. This process results in reduced LDL receptor density, decreased clearance of LDL-C, and, consequently, accumulation of LDL-C in the circulation. Thus, PCSK9 levels tend to correlate directly with LDL-C levels. In animal models, it is known that mutations that increase PCSK9 activity cause hypercholesterolemia and coronary heart disease (CHD); mutations that inactivate PCSK9 lower LDL levels and reduce CHD. PCSK9 inhibitors are therefore considered attractive potential therapeutic agents for FH, including HoFH. Among the inhibitors under development are the anti-PCSK9 antibodies (i.e. antibodies that bind to PCSK9 and prevent it binding to liver LDL receptors) evolocumab, alirocumab, bococizumab, RG7652, LY3015014, and LGT-209, of which evolocumab and alirocumab are the furthest advanced; the antisense RNAi oligonucleotide ALN-PCSsc (a GalNAc-modified second generation subcutaneously-administrable agent, awaiting approval for its first Phase 1 trial, based on ALN-PCS, which had undergone a Phase 1 trial); the pegylated adnectin BMS-962476; and others.

Evolocumab has recently been the subject of a US Biologics License Application (August 2014) and an EMA Marketing Authorization Application (September 2014), based on data from the PROFICIO program, in which evolocumab reduced LDL-C levels in hypercholesterolemic subjects more than 50%. Evolocumab has also been tested in HeFH in 331 patients in the RUTHERFORD-2 trial (Raal et al., “PCSK9 inhibition with evolocumab (AMG 145) in heterozygous familial hypercholesterolaemia (RUTHERFORD-2): a randomised, double-blind, placebo-controlled trial”, Lancet, online publication Oct. 2, 2014), using subcutaneous injection of 140 mg every 2 weeks or 420 mg every month by subcutaneous injection. Significant reductions in LDL-C were seen in both treatment groups relative to placebo; and evolocumab was said to be well tolerated, with the most common AEs occurring more frequently in the treatment groups being nasopharyngitis (9% vs. 5% for placebo) and muscle-related AEs (5% vs. 1%). Alirocumab has also been tested in hypercholesterolemia and in a placebo-controlled Phase 2 study in HeFH using subcutaneous injection at 150 mg every 2 weeks or 150, 200, or 300 mg every 4 weeks, with significant reductions seen in LDL-C (29% for 150 mg/4 weeks to 68% for 150 mg/2 weeks). Alirocumab was said to be well tolerated, with the most common reported AE being injection-site reaction. US and EU regulatory filings are reported to be expected at the end of 2014. Bococizumab has been tested in hypercholesterolemia and is under study in HeFH. A Phase 2 study in hypercholesterolemia using subcutaneous injection at 50, 100, or 150 mg twice monthly or 200 or 300 mg once monthly in 354 patients dose-ranging, double-blind, placebo-controlled study in 354 patients, with dose lowering if LDL-C was reduced to ≦25 mg/dL, showed significant reductions in LDL-C at week 12, with the greatest reductions seen with 150 mg for the twice monthly regimen and 300 mg for the once monthly regimen. The Phase 3 trial will use every 2 week dosing. ALN-PCS completed a single ascending dose Phase 1 study in hypercholesterolemic subjects, using intravenous doses between 0.015 and 0.040 mg/Kg, with a mean 70% reduction in PCSK9 at the highest dose, while ALN-PCS was said to be well tolerated. BMS-962476 has completed a single ascending dose Phase 1 study in hypercholesterolemic subjects, using subcutaneous doses of 0.01, 0.03, 0.1, and 0.3 mg/Kg and intravenous doses of 0.3 and 1.0 mg/Kg alone, and 0.1 and 0.3 mg/Kg in combination with statins. BMS-962476 was said to be well tolerated, and doses ≧0.3 mg/Kg reduced PCSK9 by at least 90%.

MBX-8025

MBX-8025 is the Compound of the Formula

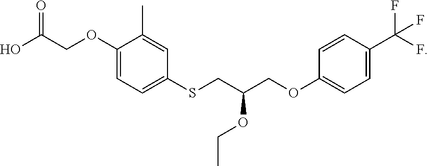 [SMILES: CCO[C@H](COc1ccc(C(F)(F)F)cc1)CSc1ccc(OCC(=O)O)c(C)c1]

MBX-8025 has the chemical name (R)-2-(4-((2-ethoxy-3-(4-(trifluoromethyl)phenoxy)propyl)thio)-2-methylphenoxy)acetic acid [IUPAC name as generated by CHEMDRAW ULTRA 12.0]. MBX-8025 and its synthesis, formulation, and use is disclosed in, for example, U.S. Pat. No. 7,301,050 (compound 15 in Table 1, Example M, claim 49), U.S. Pat. No. 7,635,718 (compound 15 in Table 1, Example M), and U.S. Pat. No. 8,106,095 (compound 15 in Table 1, Example M, claim 14). Lysine (L-lysine) salts of MBX-8025 and related compounds are disclosed in U.S. Pat. No. 7,709,682 (MBX-8025 L-lysine salt throughout the Examples, crystalline forms claimed).

MBX-8025 is an orally active, potent (2 nM) agonist of peroxisome proliferator-activated receptor-δ (PPARδ), which is also specific (>600-fold and >2500-fold compared with PPARα and PPARγ receptors). PPARδ activation stimulates fatty acid oxidation and utilization, improves plasma lipid and lipoprotein metabolism, glucose utilization, and mitochondrial respiration, and preserves stem cell homeostasis. According to U.S. Pat. No. 7,301,050, PPARδ agonists, such as MBX-8025, are suggested to treat PPARδ-mediated conditions, including “diabetes, cardiovascular diseases, Metabolic X syndrome, hypercholesterolemia, hypo-HDL-cholesterolemia, hyper-LDL-cholesterolemia, dyslipidemia, atherosclerosis, and obesity”, with dyslipidemia said to include hypertriglyceridemia and mixed hyperlipidemia.

A Phase 2 study of MBX-8025 L-lysine dihydrate salt in mixed dyslipidemia (6 groups, 30 subjects/group: once daily placebo, atorvastatin 20 mg, or MBX-8025 L-lysine dihydrate salt at 50 or 100 mg (calculated as the free acid) capsules alone or combined with atorvastatin 20 mg, for 8 weeks) has been reported by Bays et al., “MBX-8025, A Novel Peroxisome Proliferator Receptor-δ Agonist: Lipid and Other Metabolic Effects in Dyslipidemic Overweight Patients Treated with and without Atorvastatin”, J. Clin. Endocrin. Metab., 96(9), 2889-2897 (2011) and Choi et al., “Effects of the PPAR-δ agonist MBX-8025 on atherogenic dyslipidemia”, Atherosclerosis, 220, 470-476 (2012). Compared to placebo, MBX-8025 alone and in combination with atorvastatin significantly (P<0.05) reduced apoB 100 by 20-38%, LDL by 18-43%, triglycerides by 26-30%, non-HDL-C by 18-41%, free fatty acids by 16-28%, and high-sensitivity C-reactive protein by 43-72%; it raised HDL-C by 1-12% and also reduced the number of patients with the metabolic syndrome and a preponderance of small LDL particles. While MBX-8025 at 100 mg/day reduced LDL-C by 22% over the total population treated, the percentage reduction in LDL-C increased to 35% in the tertile with the highest starting LDL-C levels (187-205 mg/dL), and trend analysis on individual patient data confirmed a positive correlation between percentage reduction in LDL-C and starting LDL-C level. MBX-8025 reduced LDL-S/VS by 40-48% compared with a 25% decrease with atorvastatin; and MBX-8025 increased LDL-L by 34-44% compared with a 30% decrease with atorvastatin. MBX-8025 significantly reduced alkaline phosphatase by 32-43%, compared to reductions of only 4% in the control group and 6% in the ATV group; and significantly reduced γ-glutamyl transpeptidase by 24-28%, compared to a reduction of only 3% in the control group and an increase of 2% in the ATV group. Thus MBX-8025 corrects all three lipid abnormalities in mixed dyslipidemia—lowers TGs and LDL and raises HDL, selectively depletes small dense LDL particles (92%), reduces cardiovascular inflammation, and improves other metabolic parameters including reducing serum aminotransferases, increases insulin sensitivity (lowers HOMA-IR, fasting plasma glucose, and insulin), lowers γ-glutamyl transpeptidase and alkaline phosphatase, significantly (>2-fold) reduces the percentage of subjects meeting the criteria for metabolic syndrome, and trends towards a decrease in waist circumference and increase in lean body mass. MBX-8025 was safe and generally well-tolerated, and also reduced liver enzyme levels. As explained in US Patent Application Publication No. 2010-0152295, MBX-8025 converts LDL particle size pattern I to pattern A; and from pattern B to pattern I or A, where LDL particle size pattern B is a predominant LDL particle size of less than 25.75 nm, pattern I is a predominant LDL particle size of from 25.75 nm to 26.34 nm, and pattern A is a predominant LDL particle size of greater than 26.34 nm, where the LDL particle size is measured by gradient-gel electrophoresis.

The disclosures of the documents referred to in this application are incorporated into this application by reference.

### SUMMARY OF THE INVENTION

This invention is the treatment of homozygous familial hypercholesterolemia, comprising administration of (R)-2-(4-((2-ethoxy-3-(4-(trifluoromethyl)phenoxy)propyl)thio)-2-methyl-phenoxy)acetic acid or a salt thereof (MBX-8025 or an MBX-8025 salt); optionally in combination with an MTP inhibitor, an apoB-100 synthesis inhibitor, or a PCSK9 inhibitor.

In various aspects, this invention is:
a method of treating homozygous familial hypercholesterolemia by administering MBX-8025 or an MBX-8025 salt, optionally in combination with an MTP inhibitor, an apoB-100 synthesis inhibitor, or a PCSK9 inhibitor;
pharmaceutical compositions for treating homozygous familial hypercholesterolemia comprising MBX-8025 or an MBX-8025 salt in combination with an MTP inhibitor; and
kits for treating homozygous familial hypercholesterolemia comprising compositions comprising MBX-8025 or an MBX-8025 salt, optionally in combination with compositions containing an MTP inhibitor, an apoB-100 synthesis inhibitor, or a PCSK9 inhibitor.

The MTP inhibitor may be lomitapide or a salt thereof, or may also be SLx-4090 or JTT-130. The apoB-100 synthesis inhibitor may be mipomersen or a salt thereof. The PCSK9 inhibitor may be an anti-PCSK9 antibody such as evolocumab, alirocumab, bococizumab, RG7652, LY3015014, and LGT-209; an antisense RNAi oligonucleotide such as ALN-PCSsc; or an adnectin such as BMS-962476.

Because MBX-8025 reduces hepatic triglycerides and stimulates fatty acid oxidation resulting in a diminution of fat, its use will avoid the adverse effects of hepatic steatosis and hepatotoxicity seen with JUXTAPID and KYNAMRO. Also, because its effects, mediated by PPARδ, do not require an effective LDLR to lower LDL-C and improve other lipid parameters (an effect seen in knockout mice lacking LDLR), MBX-8025 will have a special benefit in patients with HoFH. Finally, because its effect on LDL-C reduction has been seen to increase in dyslipidemic patients with higher starting LDL-C levels, MBX-8025 is expected to be especially effective in HoFH, where starting LDL-C levels may be extremely elevated.

Because lomitapide inhibits MTP and mipomersen inhibits apoB-100 synthesis, both resulting in accumulation of hepatic fat, while MBX-8025 reduces hepatic triglycerides and stimulates fatty acid oxidation resulting in a diminution of fat, combination therapy with MBX-8025 and lomitapide or mipomersen will result in ameliorating the adverse reactions to the lomitapide or mipomersen, thereby reducing safety concerns, while preserving the benefits of the treatment with each compound. Similar effects are expected with other MTP inhibitors and apoB-100 synthesis inhibitors.

Preferred embodiments of this invention are characterized by the specification and by the features of claims 1 to 22 of this application as filed.

### DETAILED DESCRIPTION OF THE INVENTION

Definitions

“Homozygous familial hypercholesterolemia” or “HoFH” is described in paragraphs [0003] through [0005].

“MBX-8025” and its salts, are described in paragraphs [0029] through [0032].

“MTP inhibitors”, including lomitapide and its salts, are described in paragraphs [0008] through [0016]; “apoB-100 synthesis inhibitors”, including mipomersen and its salts, are described in paragraphs [0017] through [0024]; and “PCSK9 inhibitors, including anti-PCSK9 antibodies such as evolocumab, alirocumab, bococizumab, RG7652, LY3015014, and LGT-209; antisense RNAi oligonucleotides such as ALN-PCSsc; and adnectins such as BMS-962476, are described in paragraphs [0026] through [0028], respectively.

A “therapeutically effective amount” of MBX-8025 or an MBX-8025 salt means that amount which, when administered to a human for treating HoFH, is sufficient to effect treatment for HoFH. A “therapeutically effective amount” of each of (MBX-8025 or an MBX-8025 salt) and an MTP inhibitor, an apoB-100 synthesis inhibitor, or a PCSK9 inhibitor means that amount which, when administered in combination therapy to a human for treating HoFH, is sufficient to effect treatment for HoFH.

“Treating” or “treatment” of HoFH in a human includes one or more of:
(1) preventing or reducing the risk of developing HoFH, i.e., causing at least one of the clinical symptoms of HoFH not to develop in a subject who may be predisposed to HoFH but who does not yet experience or display symptoms of the HoFH (i.e. prophylaxis);
(2) inhibiting HoFH, i.e., arresting or reducing the development of HoFH or at least one of its clinical symptoms; and
(3) relieving HoFH, i.e., causing regression, reversal, or amelioration of HoFH or reducing the number, frequency, duration or severity of a least one of its clinical symptoms.
   The therapeutically effective amount for a particular subject varies depending upon the health and physical condition of the subject to be treated, the extent of HoFH, the assessment of the medical situation, and other relevant factors. It is expected that the therapeutically effective amount will fall in a relatively broad range, as discussed below, and that this amount can be determined through routine trial based on the ordinary skill in the art and the guidance of this application.

Salts (for example, pharmaceutically acceptable salts) of MBX-8025 and of the MTP inhibitor, apoB-100 synthesis inhibitor, or PCSK9 inhibitor are included in this invention and are useful in the compositions, methods, and uses described in this application. These salts are preferably formed with pharmaceutically acceptable acids and bases. See, for example, “Handbook of Pharmaceutically Acceptable Salts”, Stahl and Wermuth, eds., Verlag Helvetica Chimica Acta, Zurich, Switzerland, for an extensive discussion of pharmaceutical salts, their selection, preparation, and use. Unless the context requires otherwise, reference to MBX-8025 and other compounds is a reference both to the compound and to its salts.

Because MBX-8025 contains a carboxyl group, it may form salts when the acidic proton present reacts with inorganic or organic bases. Typically the MBX-8025 is treated with an excess of an alkaline reagent, such as hydroxide, carbonate or alkoxide, containing an appropriate cation. Cations such as Na⁺, K⁺, Ca²⁺, Mg²⁺, and NH₄⁺ are examples of cations present in pharmaceutically acceptable salts. Suitable inorganic bases, therefore, include calcium hydroxide, potassium hydroxide, sodium carbonate and sodium hydroxide. Salts may also be prepared using organic bases, such as salts of primary, secondary and tertiary amines, substituted amines including naturally-occurring substituted amines, and cyclic amines including isopropylamine, trimethylamine, diethylamine, triethylamine, tripropylamine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, N-ethylpiperidine, and the like. As noted in paragraph [0032], MBX-8025 has been studied in clinical trials as its L-lysine dihydrate salt, and MBX-8025 has also been studied in clinical trials as its calcium salt.

Because lomitapide contains a basic group, the piperidine amino group, it may be prepared as an acid addition salt. Acid addition salts are prepared in a standard manner in a suitable solvent from lomitapide and an excess of an acid, such as hydrochloric acid, hydrobromic acid, sulfuric acid (giving the sulfate and bisulfate salts), nitric acid, phosphoric acid and the like, and organic acids such as acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, salicylic acid, 4-toluenesulfonic acid, hexanoic acid, heptanoic acid, cyclopentanepropionic acid, lactic acid, 2-(4-hydroxybenzoyl)benzoic acid, 1,2-ethanedisulfonic acid, 2-hydroxyethanesulfonic acid, benzenesulfonic acid, 4-chlorobenzenesulfonic acid, 2-naphthalenesulfonic acid, camphorsulfonic acid, 4-methylbicyclo[2.2.2.]oct-2-ene-1-carboxylic acid, glucoheptonic acid, gluconic acid, 3-hydroxy-2-naphthoic acid, 4,4′-methylenebis(3-hydroxy-2-naphthoic)acid, 3-phenylpropionic acid, trimethylacetic acid, tert-butylacetic acid, laurylsulfuric acid, glucuronic acid, glutamic acid, stearic acid, muconic acid, and the like. As noted in paragraph [0012], lomitapide is currently formulated as its mesylate salt in JUXTAPID.

Because mipomersen contains acidic groups, the thiolate groups, it may form salts when the acidic protons present reacts with inorganic or organic bases. As noted in paragraph [0020], mipomersen is currently formulated as its sodium salt in KYNAMRO.

“Combination therapy” with MBX-8025 and an MTP inhibitor, an apoB-100 synthesis inhibitor, or a PCSK9 inhibitor means administration of MBX-8025 and an MTP inhibitor, an apoB-100 synthesis inhibitor, or a PCSK9 inhibitor during the course of treatment of HoFH. Such combination therapy may involve administration of an MTP inhibitor, an apoB-100 synthesis inhibitor, or a PCSK9 inhibitor before, during, and/or after administration of MBX-8025, such that therapeutically effective levels of each of the compounds are maintained. Because MBX-8025 and lomitapide are each administered orally once/day, and because lomitapide is indicated to be taken at least 2 hours after the evening meal, it may be convenient to administer MBX-8025 at the same time as lomitapide is administered. Combination therapy also includes the administration of a single dosage form (e.g. a capsule) containing both MBX-8025 and lomitapide. Similar dosing is expectable for other orally active MTP inhibitors. Because the other compounds, apoB-100 synthesis inhibitors and PCSK9 inhibitors, including mipomersen, are administered by injection less frequently, such as once/week for mipomersen, and every 2 or 4 weeks for the PCSK9 antibodies, it may be convenient to administer these compounds, on the day of the week selected for administration of mipomersen, at the same time as the MBX-8025 is administered.

“Comprising” or “containing” and their grammatical variants are words of inclusion and not of limitation and mean to specify the presence of stated components, groups, steps, and the like but not to exclude the presence or addition of other components, groups, steps, and the like. Thus “comprising” does not mean “consisting of”, “consisting substantially of” or “consisting only of” and, for example, a formulation “comprising” a compound must contain that compound but also may contain other active ingredients and/or excipients.

Formulation and Administration

The MBX-8025, and optionally the MTP inhibitor, the apoB-100 synthesis inhibitor, or the PCSK9 inhibitor, may be administered by any route suitable to the subject being treated and the nature of the subject's condition. Routes of administration include administration by injection, including intravenous, intraperitoneal, intramuscular, and subcutaneous injection, by transmucosal or transdermal delivery, through topical applications, nasal spray, suppository and the like or may be administered orally. Formulations may optionally be liposomal formulations, emulsions, formulations designed to administer the drug across mucosal membranes or transdermal formulations. Suitable formulations for each of these methods of administration may be found, for example, in “Remington: The Science and Practice of Pharmacy”, 20th ed., Gennaro, ed., Lippincott Williams & Wilkins, Philadelphia, Pa., U.S.A. Because both MBX-8025 and lomitapide are orally available, typical formulations will be oral, and typical dosage forms of each of the components of the combination therapy, or of the two components together, will be tablets or capsules for oral administration. As mentioned in paragraph [0012], lomitapide is currently formulated as capsules; and as mentioned in paragraph [0032], MBX-8025 has been formulated in capsules for clinical trials. As mentioned in paragraph [0020], mipomersen sodium is currently formulated as a solution for subcutaneous injection, dispensed either in a single-use vial or a single-use prefilled syringe. The PCSK9 inhibitors are all formulated as solutions for injection, typically for subcutaneous injection.

Depending on the intended mode of administration, the pharmaceutical compositions may be in the form of solid, semi-solid or liquid dosage forms, preferably in unit dosage form suitable for single administration of a precise dosage. In addition to an effective amount of the MBX-8025, the MTP inhibitor, the apoB-100 synthesis inhibitor, and the PCSK9 inhibitor, the compositions may contain suitable pharmaceutically-acceptable excipients, including adjuvants which facilitate processing of the active compounds into preparations which can be used pharmaceutically. “Pharmaceutically acceptable excipient” refers to an excipient or mixture of excipients which does not interfere with the effectiveness of the biological activity of the active compound(s) and which is not toxic or otherwise undesirable to the subject to which it is administered.

For solid compositions, conventional excipients include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by dissolving, dispersing, etc., an active compound as described herein and optional pharmaceutical adjuvants in water or an aqueous excipient, such as, for example, water, saline, aqueous dextrose, and the like, to form a solution or suspension. If desired, the pharmaceutical composition to be administered may also contain minor amounts of nontoxic auxiliary excipients such as wetting or emulsifying agents, pH buffering agents and the like, for example, sodium acetate, sorbitan monolaurate, triethanolamine sodium acetate, triethanolamine oleate, etc.

For oral administration, the composition will generally take the form of a tablet or capsule, or it may be an aqueous or nonaqueous solution, suspension or syrup. Tablets and capsules are preferred oral administration forms. Tablets and capsules for oral use will generally include one or more commonly used excipients such as lactose and corn starch. Lubricating agents, such as magnesium stearate, are also typically added. When liquid suspensions are used, the active agent may be combined with emulsifying and suspending excipients. If desired, flavoring, coloring and/or sweetening agents may be added as well. Other optional excipients for incorporation into an oral formulation include preservatives, suspending agents, thickening agents, and the like.

Typically, a pharmaceutical composition of MBX-8025 is packaged in a container with a label, or instructions, or both, indicating use of the pharmaceutical composition in the treatment of HoFH. Typically, a pharmaceutical composition of the combination of MBX-8025 and an MTP inhibitor such as lomitapide, or a kit comprising separate compositions of MBX-8025 and of an MTP inhibitor, an apoB-100 synthesis inhibitor, or a PCSK9 inhibitor, is packaged in a container with a label, or instructions, or both, indicating use of the pharmaceutical composition or kit in the treatment of HoFH.

A suitable amount of MBX-8025 (calculated as the free acid) for oral dosing when administered alone (i.e. not administered in combination with an MTP inhibitor, an apoB-100 synthesis inhibitor, or a PCSK9 inhibitor: HoFH patients may well be taking other lipid-lowering therapies in addition to the compounds discussed in this application) will be 20-200 mg/day, preferably 50-200 mg/day. That is, a suitable amount of MBX-8025 for oral dosing will be similar to the amounts employed in clinical trials; though it is possible that the therapeutically effective amount may be higher in severe cases of HoFH.

When MBX-8025 and an MTP inhibitor, an apoB-100 synthesis inhibitor, or a PCSK9 inhibitor are used in combination therapy, a suitable amount of MBX-8025 (calculated as the free acid) for oral dosing will be 20-200 mg/day, preferably 50-200 mg/day; and suitable amounts of the MTP inhibitor, apoB-100 synthesis inhibitor, or PCSK9 inhibitor will be similar to the amounts approved or used in clinical trials, as described in paragraphs [0007] through [0028]. Thus, for example, a suitable amount of lomitapide (calculated as the mesylate salt) for oral dosing will be 10-100 mg/day, preferably between 20-80 mg/day, especially 30-60 mg/day, typically administered once/day; and a suitable amount of mipomersen (calculated as the sodium salt) for subcutaneous dosing will be 100-300 mg/week, preferably 200 mg/week, typically administered once/week. That is, suitable amounts of MBX-8025 and the MTP inhibitor, apoB-100 synthesis inhibitor, or PCSK9 inhibitor to achieve a therapeutically effective amount of the combination therapy will be similar to the amounts employed in clinical trials (and currently marketed, in the case of lomitapide and mipomersen). However, it is possible that the therapeutically effective amounts of either may be less in combination therapy than when used as monotherapy because each of MBX-8025, MTP inhibitors, apoB-100 synthesis inhibitors, and PCSK9 inhibitors is useful in lowering cholesterol, and it is also possible that the combination therapy, by the MBX-8025 reducing the adverse effects of MTP inhibitor (e.g. lomitapide) monotherapy or apoB-100 synthesis inhibitor (e.g. mipomersen) monotherapy, may permit the use of a greater dose of an MTP inhibitor or apoB-100 synthesis inhibitor (e.g. lomitapide or mipomersen) than is currently approved in lomitapide or mipomersen monotherapy. Typical dosage forms for MBX-8025 and lomitapide will contain a single daily dose.

A person of ordinary skill in the art of the treatment of HoFH will be able to ascertain a therapeutically effective amount of MBX-8025 when used alone, or the therapeutically effective amounts of MBX-8025 and an MTP inhibitor, an apoB-100 synthesis inhibitor, or a PCSK9 inhibitor, when used in combination therapy, for a particular patient and stage of HoFH to achieve a therapeutically effective amount without undue experimentation and in reliance upon personal knowledge and the disclosure of this application.

### EXAMPLES

### Example 1

### Study with MBX-8025

Subjects with HoFH (diagnosed either by genetic testing or by an untreated LDL-C >500 mg/dL and early appearance of xanthoma or LDL-C levels consistent with HeFH in both parents), on maximally-tolerated lipid-lowering therapy, are treated with MBX-8025 L-lysine dihydrate salt at a dose of 50, 100, or 200 mg/day (as MBX-8025 free acid). Subjects are permitted their usual other medications, including lipid-lowering treatments. The subjects are assessed before the study, and at intervals during the study, such as every 4 weeks during the study and 4 weeks after the last dose of the MBX-8025 therapy, for safety and pharmacodynamic evaluations. MRIs of the subjects' livers are taken every 4 weeks during the study and 4 weeks after study completion, to determine hepatic fat. At each visit, after a 12-hour fast, blood is drawn and urine collected; and a standard metabolic panel, complete blood count, and standard urinalysis are performed. Blood is analyzed for TC, HDL-C, LDL-C, VLDL-C, TG, and apoB. The subjects also maintain health diaries, which are reviewed at each visit.

MBX-8025 causes dose-dependent lowering of TC, LDL-C, VLDL-C, TG and apoB, and raising of HDL-C.

### Example 2

### Dose Escalation Study with MBX-8025 and Lomitapide

Subjects with HoFH(diagnosed either by genetic testing or by an untreated LDL-C >500 mg/dL and early appearance of xanthoma or LDL-C levels consistent with HeFH in both parents), on maximally-tolerated lipid-lowering therapy, are treated with MBX-8025 L-lysine dihydrate salt at a dose of 50, 100, or 200 mg/day (as MBX-8025 free acid) in combination with escalating doses of lomitapide (lomitapide mesylate doses of 5, 10, 20, 40, and 60 mg/day each for 4 weeks). The subjects are instructed to maintain a low-fat diet (<20% energy from fat) and to take dietary supplements that provide approximately 400 IU vitamin E, 210 mg α-linolenic acid, 200 mg linoleic acid, 110 mg eicosapentenoic acid, and 80 mg docosahexaenoic acid per day; and are permitted their usual other medications, although other lipid-lowering treatments are suspended. The subjects are assessed before the study, and at intervals during the study, such as every 1, 2, and 4 weeks after the start of a new dose and 4 weeks after the last dose of the combination therapy, for safety and pharmacodynamic evaluations. MRIs of the subjects' livers are taken after 4 weeks at each dose, and 4 weeks after study completion, to determine hepatic fat. At each visit, after a 12-hour fast, blood is drawn and urine collected; and a standard metabolic panel, complete blood count, and standard urinalysis are performed. Blood is analyzed for TC, HDL-C, TG, VLDL-C, LDL-C and apoB. The subjects also maintain health diaries, which are reviewed at each visit.

The combination of MBX-8025 and lomitapide causes dose-dependent lowering of TC, LDL-C, VLDL-C, TG, and apoB, and raising of HDL-C, while the hepatic fat increases usually caused by lomitapide monotherapy are reduced.

### Example 3

### Study with MBX-8025 and Mipomersen

Subjects with HoFH(diagnosed either by genetic testing or by an untreated LDL-C >500 mg/dL and early appearance of xanthoma or LDL-C levels consistent with HeFH in both parents), on maximally-tolerated lipid-lowering therapy, are treated with MBX-8025 L-lysine dihydrate salt at a dose of 50, 100, or 200 mg/day (as MBX-8025 free acid) in combination with mipomersen sodium doses of 200 mg/week (or 160 mg/week for subjects weighing less than 50 Kg). The subjects are instructed to maintain their usual diet and medications. The subjects are assessed before the study, and at intervals during the study, such as every 2 weeks for the first month, every 4 weeks thereafter, and 4 weeks after the last dose of the combination therapy, for safety and pharmacodynamic evaluations. MRIs of the subjects' livers are taken at baseline and 4 weeks after study completion, to determine hepatic fat. At each visit, after a 12-hour fast, blood is drawn and urine collected; and a standard metabolic panel, complete blood count, and standard urinalysis are performed. Blood is analyzed for TC, HDL-C, TG, VLDL-C, LDL-C, and apoB, and for serum aminotransferases. The subjects also maintain health diaries, which are reviewed at each visit.

The combination of MBX-8025 and mipomersen causes dose-dependent lowering of TC, LDL-C, VLDL-C, TG, and apoB, and raising of HDL-C, while the hepatic fat increases usually caused by mipomersen monotherapy are reduced.

Similar studies may be conducted with MBX-8025 and other MTP inhibitors, other apoB-100 synthesis inhibitors, or PCSK9 inhibitors; and a reduction in LDL-C is expectable.

While this invention has been described in conjunction with specific embodiments and examples, it will be apparent to a person of ordinary skill in the art, having regard to that skill and this disclosure, that equivalents of the specifically disclosed materials and methods will also be applicable to this invention; and such equivalents are intended to be included within the following claims.

1. A method of treating homozygous familial hypercholesterolemia by administering (R)-2-(4-((2-ethoxy-3-(4-(trifluoromethyl)phenoxy)propyl)thio)-2-methylphenoxy)acetic acid or a salt thereof; optionally in combination with an MTP inhibitor, an apoB-100 synthesis inhibitor, or a PCSK9 inhibitor.

2.-22. (canceled)

23. The method of claim 1 where the (R)-2-(4-((2-ethoxy-3-(4-(trifluoromethyl)-phenoxy)propyl)thio)-2-methylphenoxy)acetic acid or a salt thereof is (R)-2-(4-((2-ethoxy-3-(4-(trifluoromethyl)phenoxy)propyl)thio)-2-methylphenoxy)acetic acid L-lysine dihydrate.

24. The method of claim 1 where the amount administered of the (R)-2-(4-((2-ethoxy-3-(4-(trifluoromethyl)phenoxy)propyl)thio)-2-methylphenoxy)acetic acid or a salt thereof (when calculated as the free acid) is 20-200 mg/day.

25. The method of claim 1 where the (R)-2-(4-((2-ethoxy-3-(4-(trifluoromethyl)-phenoxy)propyl)thio)-2-methylphenoxy)acetic acid or a salt thereof is administered once/day.

26. The method of claim 1 where the (R)-2-(4-((2-ethoxy-3-(4-(trifluoromethyl)-phenoxy)propyl)thio)-2-methylphenoxy)acetic acid or a salt thereof is administered alone.

27. The method of claim 1 where the (R)-2-(4-((2-ethoxy-3-(4-(trifluoromethyl)-phenoxy)propyl)thio)-2-methylphenoxy)acetic acid or a salt thereof is administered in combination with an MTP inhibitor.

28. The method of claim 27 where the MTP inhibitor is lomitapide or a salt thereof, SLx-4090, or JTT-130.

29. The method of claim 28 where the MTP inhibitor is lomitapide or a salt thereof.

30. The method of claim 29 where the MTP inhibitor is lomitapide mesylate.

31. The method of claim 30 where the amount administered of the lomitapide mesylate is 10-100 mg/day, administered once/day; the (R)-2-(4-((2-ethoxy-3-(4-(trifluoromethyl)phenoxy)propyl)thio)-2-methylphenoxy)acetic acid or a salt thereof is (R)-2-(4-((2-ethoxy-3-(4-(trifluoromethyl)phenoxy)propyl)thio)-2-methylphenoxy)acetic acid L-lysine dihydrate, and the amount administered of the (R)-2-(4-((2-ethoxy-3-(4-(trifluoromethyl))propyl)thio)-2-methylphenoxy)acetic acid L-lysine dihydrate (when calculated as the free acid) is 20-200 mg/day, administered once/day.

32. The method of claim 1 where the (R)-2-(4-((2-ethoxy-3-(4-(trifluoromethyl)-phenoxy)propyl)thio)-2-methylphenoxy)acetic acid or a salt thereof is administered in combination with an apoB-100 synthesis inhibitor.

33. The method of claim 32 where the apoB-100 synthesis inhibitor is mipomersen or a salt thereof.

34. The method of claim 33 where the apoB-100 synthesis inhibitor is mipomersen sodium.

35. The method of claim 34 where the amount administered of the mipomersen sodium is 300 mg, administered once/week; the (R)-2-(4-((2-ethoxy-3-(4-(trifluoromethyl)-phenoxy)propyl)thio)-2-methylphenoxy)acetic acid or a salt thereof is (R)-2-(4-((2-ethoxy-3-(4-(trifluoromethyl)phenoxy)propyl)thio)-2-methylphenoxy)acetic acid L-lysine dihydrate, and the amount administered of the (R)-2-(4-((2-ethoxy-3-(4-(trifluoromethyl)phenoxy)propyl)thio)-2-methylphenoxy)acetic acid L-lysine dihydrate (when calculated as the free acid) is 20-200 mg/day, administered once/day.

36. The method of claim 1 where the (R)-2-(4-((2-ethoxy-3-(4-(trifluoromethyl)-phenoxy)propyl)thio)-2-methylphenoxy)acetic acid or a salt thereof is administered in combination with a PCSK9 inhibitor.

37. The method of claim 36 where the PCSK9 inhibitor is evolocumab, alirocumab, bococizumab, RG7652, LGT-209, LY3015014, ALN-PCSsc, or BMS-962476.

38. The method of claim 37 where the PCSK9 inhibitor is evolocumab.

39. The method of claim 37 where the PCSK9 inhibitor is alirocumab.

40. The method of claim 37 where the PCSK9 inhibitor is bococizumab.
